(19) United States
(12) Patent Application Publication
(10) Pub. No.: US 2013/0317254 A1
(43) Pub. Date: Nov. 28, 2013

(54) Process for Producing Acrylic Acids and Acrylates

(71) Applicants: Ilias S. Kotsianis, Houston, TX (US); Tianshu Pan, Houston, TX (US); Craig J. Peterson, Houston, TX (US); R. Jay Warner, Houston, TX (US); Josefina T. Chapman, Houston, TX (US)

(72) Inventors: Ilias S. Kotsianis, Houston, TX (US); Tianshu Pan, Houston, TX (US); Craig J. Peterson, Houston, TX (US); R. Jay Warner, Houston, TX (US); Josefina T. Chapman, Houston, TX (US)

(73) Assignee: Celanese International Corporation, Irving, TX (US)

(21) Appl. No.: 13/480,620

(22) Filed: May 25, 2012

(51) Int. Cl. C07C 51/46; C07C 51/347

(52) U.S. Cl. 562/599; 562/600

(57) ABSTRACT

In one embodiment, the invention is to a process for producing an acrylate product. The process comprises the step of providing a crude product stream comprising the acrylate product and an alkylenating agent. The process further comprises the step of separating at least a portion of the crude product stream into an intermediate acrylate product stream and an alkylenating stream. The process further comprises separating at least a portion of the intermediate acrylate product stream by azeotropic distillation in the presences of an entrainer to form a stream containing acrylate product and a stream containing entrainer.

### FIELD OF THE INVENTION

The present invention relates generally to the production of acrylic acid. More specifically, the present invention relates to the production of crude acrylic acid via the condensation of acetic acid and formaldehyde and the subsequent purification thereof.

### BACKGROUND OF THE INVENTION

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes: (1) the reaction of acetylene with water and carbon monoxide; and/or (2) the reaction of an alcohol and carbon monoxide, in the presence of an acid, e.g., hydrochloric acid, and nickel tetracarbonyl, to yield a crude product comprising the acrylate ester as well as hydrogen and nickel chloride. Another conventional process involves the reaction of ketene (often obtained by the pyrolysis of acetone or acetic acid) with formaldehyde, which yields a crude product comprising acrylic acid and either water (when acetic acid is used as a pyrolysis reactant) or methane (when acetone is used as a pyrolysis reactant). These processes have become obsolete for economic, environmental, or other reasons.

More recent acrylic acid production processes have relied on the gas phase oxidation of propylene, via acrolein, to form acrylic acid. The reaction can be carried out in single- or two-step processes but the latter is favored because of higher yields. The oxidation of propylene produces acrolein, acrylic acid, acetaldehyde and carbon oxides. Acrylic acid from the primary oxidation can be recovered while the acrolein is fed to a second step to yield the crude acrylic acid product, which comprises acrylic acid, water, small amounts of acetic acid, as well as impurities such as furfural, acrolein, and propionic acid. Purification of the crude product may be carried out by azeotropic distillation. Although this process may show some improvement over earlier processes, this process suffers from production and/or separation inefficiencies. In addition, this oxidation reaction is highly exothermic and, as such, creates an explosion risk. As a result, more expensive reactor design and metallurgy are required. Also, the cost of propylene is often prohibitive.

The aldol condensation reaction of formaldehyde and acetic acid and/or carboxylic acid esters has been disclosed in literature. This reaction forms acrylic acid and is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). The acetic acid conversions in these reactions, however, may leave room for improvement. Although this reaction is disclosed, there has been little if any disclosure relating to separation schemes that may be employed to effectively provide purified acrylic acid from the aldol condensation crude product.

Thus, the need exists for processes for producing purified acrylic acid and, in particular, for separation schemes to effectively purify unique aldol condensation crude products to form the purified acrylic acid.

The references mentioned above are hereby incorporated by reference.

### BRIEF DESCRIPTION OF DRAWINGS

The invention is described in detail below with reference to the appended drawings, wherein like numerals designate similar parts.

FIG. 1 is a process flowsheet showing an acrylic acid reaction/separation system in accordance with an embodiment of the present invention.

FIG. 2 is a schematic diagram of an acrylic acid reaction/separation system in accordance with one embodiment of the present invention.

### SUMMARY OF THE INVENTION

In one embodiment, the invention is to a process for producing acrylic acid, methacrylic acid, and/or the salts and esters thereof. Preferably, the inventive process yields an acrylic acid product. The process comprises the step of providing a crude product stream comprising acrylate products, acetic acid and an alkylenating agent. The process further comprises the step of separating at least a portion of the crude product stream into an intermediate acrylate product stream and an alkylenating stream, wherein the intermediate acrylate product stream comprises acrylate product. The process further comprises the step of separating at least a portion of the intermediate acrylate product stream by azeotropic distillation in the presence of an entrainer to form a first distillate comprising entrainer and acetic acid and a first residue comprising acrylate product, entrainer, and acetic acid. The process further comprises recovering acrylate product from the first residue.

In another embodiment, the inventive process comprises the step of alkylenating acetic acid from an acetic acid feed stream in a reactor to form a crude acrylate product stream comprising acrylate product, an alkylenating agent and acetic acid. The process further comprises the step of separating the crude acrylate product stream into an intermediate acrylate product stream and an alkylenating stream, wherein the intermediate acrylate product stream comprises acrylate product. The process further comprises the step of separating at least a portion of the intermediate acrylate product stream using azeotropic distillation in a first column in the presence of one or more entrainers to form a first distillate comprising entrainer and acetic acid, and a first residue comprising acrylate product, entrainer, and acetic acid. The process further comprises the step of separating at least a portion of the first residue in a second column to form a second distillate comprising acetic acid and entrainer and a second residue comprising acrylate product. The process further comprises the step of recovering acrylate product from the second residue.

In another embodiment, the process of the present invention comprises the step of providing a crude product stream comprising the acrylate product, acetic acid and an alkylenating agent. The process further comprises the step of separating at least a portion of the crude product stream by azeotropic distillation in the presence of an entrainer to form a first distillate comprising entrainer and acetic acid and a first residue comprising acrylate product, entrainer, and acetic acid. The process further comprises the step of recovering acrylate product from the first residue.

In another embodiment, the process of the present invention comprises the step of providing a crude product stream comprising the acrylate product and an alkylenating agent. The process further comprises the step of diluting the crude product stream with one or more entrainers to prevent the polymerization of the acrylate product. The process further comprises the step of separating at least a portion of the diluted crude product stream and recovering the acrylate product. Preferably, the acrylate product comprises less than 5 wt. % alkylenating agent.

### DETAILED DESCRIPTION OF THE INVENTION

### Introduction

Production of unsaturated carboxylic acids such as acrylic acid and methacrylic acid and the ester derivatives thereof via most conventional processes has been limited by economic and environmental constraints. In the interest of finding a new reaction path, the aldol condensation reaction of acetic acid and an alkylenating agent, e.g., formaldehyde, has been investigated. This reaction may yield a unique crude product that comprises, inter alia, a higher amount of (residual) formaldehyde, which is generally known to add unpredictability and problems to separation schemes. Although the aldol condensation reaction of acetic acid and formaldehyde is known, there has been little if any disclosure relating to separation schemes that may be employed to effectively purify the unique crude product that is produced. Other conventional reactions, e.g., propylene oxidation or ketene/formaldehyde, do not yield crude products that comprises higher amounts of formaldehyde. The primary reactions and the side reactions in propylene oxidation do not create formaldehyde. In the reaction of ketene and formaldehyde, a two-step reaction is employed and the formaldehyde is confined to the first stage. Also, the ketene is highly reactive and converts substantially all of the reactant formaldehyde. As a result of these features, very little, if any, formaldehyde remains in the crude product exiting the reaction zone. Because no formaldehyde is present in crude products formed by these conventional reactions, the separation schemes associated therewith have not addressed the problems and unpredictability that accompany crude products that have higher formaldehyde content.

### Crude Acrylate Product

In one embodiment, the present invention is to a process for producing acrylic acid, methacrylic acid, and/or the salts and esters thereof. As used herein, acrylic acid, methacrylic acid, and/or the salts and esters thereof, collectively or individually, may be referred to as “acrylate products.” The use of the terms acrylic acid, methacrylic acid, or the salts and esters thereof, individually, does not exclude the other acrylate products, and the use of the term acrylate product does not require the presence of acrylic acid, methacrylic acid, and the salts and esters thereof.

The inventive process, in one embodiment, includes the step of providing a crude product stream comprising the acrylic acid and/or other acrylate products. The crude product stream of the present invention, unlike most conventional acrylic acid-containing crude products, further comprises a significant portion of at least one alkylenating agent. Preferably, the at least one alkylenating agent is formaldehyde. For example, the crude product stream may comprise at least 0.5 wt. % alkylenating agent(s), e.g., at least 1 wt. %, at least 5 wt. %, at least 7 wt. %, at least 10 wt. %, or at least 25 wt. %. In terms of ranges, the crude product stream may comprise from 0.5 wt. % to 50 wt. % alkylenating agent(s), e.g., from 1 wt. % to 45 wt. %, from 1 wt. % to 25 wt. %, from 1 wt. % to 10 wt. %, or from 5 wt. % to 10 wt. %. In terms of upper limits, the crude product stream may comprise less than 50 wt. % alkylenating agent(s), e.g., less than 45 wt. %, less than 25 wt. %, or less than 10 wt. %.

In one embodiment, the crude product stream of the present invention further comprises water. For example, the crude product stream may comprise less than 60 wt. % water, e.g., less than 50 wt. %, less than 40 wt. %, or less than 30 wt. %. In terms of ranges, the crude product stream may comprise from 1 wt. % to 60 wt. % water, e.g., from 5 wt. % to 50 wt. %, from 10 wt. % to 40 wt. %, or from 15 wt. % to 40 wt. %. In terms of upper limits, the crude product stream may comprise at least 1 wt. % water, e.g., at least 5 wt. %, at least 10 wt. %, or at least 15 wt. %.

In one embodiment, the crude product stream of the present invention comprises very little, if any, of the impurities found in most conventional acrylic acid crude product streams. For example, the crude product stream of the present invention may comprise less than 1000 wppm of such impurities (either as individual components or collectively), e.g., less than 500 wppm, less than 100 wppm, less than 50 wppm, or less than 10 wppm. Exemplary impurities include acetylene, ketene, beta-propiolactone, higher alcohols, e.g., C₂₊, C₃₊, or C₄₊, and combinations thereof. Importantly, the crude product stream of the present invention comprises very little, if any, furfural and/or acrolein. In one embodiment, the crude product stream comprises substantially no furfural and/or acrolein, e.g., no furfural and/or acrolein. In one embodiment, the crude product stream comprises less than 500 wppm acrolein, e.g., less than 100 wppm, less than 50 wppm, or less than 10 wppm. In one embodiment, the crude product stream comprises less than 500 wppm furfural, e.g., less than 100 wppm, less than 50 wppm, or less than 10 wppm. Furfural and acrolein are known to act as detrimental chain terminators in acrylic acid polymerization reactions. Also, furfural and/or acrolein are known to have adverse effects on the color of purified product and/or to subsequent polymerized products.

In addition to the acrylic acid and the alkylenating agent, the crude product stream may further comprise acetic acid, water, propionic acid, and light ends such as oxygen, nitrogen, carbon monoxide, carbon dioxide, methanol, methyl acetate, methyl acrylate, acetaldehyde, hydrogen, and acetone. Exemplary compositional data for the crude product stream are shown in Table 1. Components other than those listed in Table 1 may also be present in the crude product stream.

TABLE 1
CRUDE ACRYLATE PRODUCT STREAM COMPOSITIONS

| | Conc. | Conc. | Conc. | Conc. |
|---|---|---|---|---|
| Component | (wt. %) | (wt. %) | (wt. %) | (wt. %) |
| Acrylic Acid | 1 to 75 | 1 to 50 | 5 to 50 | 10 to 40 |
| Alkylenating Agent(s) | 0.5 to 50 | 1 to 45 | 1 to 25 | 1 to 10 |
| Acetic Acid | 1 to 90 | 1 to 70 | 5 to 50 | 10 to 50 |
| Water | 1 to 60 | 5 to 50 | 10 to 40 | 15 to 40 |
| Propionic Acid | 0.01 to 10 | 0.1 to 10 | 0.1 to 5 | 0.1 to 1 |
| Oxygen | 0.01 to 10 | 0.1 to 10 | 0.1 to 5 | 0.1 to 1 |
| Nitrogen | 0.1 to 20 | 0.1 to 10 | 0.5 to 5 | 0.5 to 4 |
| Carbon Monoxide | 0.01 to 10 | 0.1 to 10 | 0.1 to 5 | 0.5 to 3 |
| Carbon Dioxide | 0.01 to 10 | 0.1 to 10 | 0.1 to 5 | 0.5 to 3 |
| Other Light Ends | 0.01 to 10 | 0.1 to 10 | 0.1 to 5 | 0.5 to 3 |

### Separation

The unique crude product stream of the present invention may be separated in a separation zone to form a final product, e.g., a final acrylic acid product. In one embodiment, the inventive process comprises the step of separating at least a portion of the crude product stream to form an alkylenating agent stream and an intermediate product stream. This separating step may be referred to as an “alkylenating agent split.” In one embodiment, the alkylenating agent stream comprises significant amounts of alkylenating agent(s). For example, the alkylenating agent stream may comprise at least 1 wt % alkylenating agent(s), e.g., at least 5 wt %, at least 10 wt %, at least 15 wt %, or at least 25 wt %. In terms of ranges, the alkylenating stream may comprise from 1 wt % to 75 wt % alkylenating agent(s), e.g., from 3 wt % to 50 wt %, from 3 wt % to 25 wt %, or from 10 wt % to 20 wt %. In terms of upper limits, the alkylenating stream may comprise less than 75 wt % alkylenating agent(s), e.g. less than 50 wt % or less than 40 wt %. In preferred embodiments, the alkylenating agent is formaldehyde.

As noted above, the presence of alkylenating agent in the crude product stream adds unpredictability and problems to separation schemes. Without being bound by theory, it is believed that formaldehyde reacts in many side reactions with water to form by-products. The following side reactions are exemplary.

As noted above, the presence of alkylenating agent in the crude product stream adds unpredictability and problems to separation schemes. Without being bound by theory, it is believed that formaldehyde reacts in many side reactions with water to form by-products. The following side reactions are exemplary.

CH₂O+H₂O→HOCH₂OH

HO(CH₂O)ᵢ₋₁H+HOCH₂OH→HO(CH₂O)ᵢH+H₂O for i>1

Without being bound by theory, it is believed that, in some embodiments, as a result of these reactions, the alkylenating agent, e.g., formaldehyde, acts as a “light” component at higher temperatures and as a “heavy” component at lower temperatures. The reaction(s) are exothermic. Accordingly, the equilibrium constant increases as temperature decreases and decreases as temperature increases. At lower temperatures, the larger equilibrium constant favors methylene glycol and oligomer production and formaldehyde becomes limited, and, as such, behaves as a heavy component. At higher temperatures, the smaller equilibrium constant favors formaldehyde production and methylene glycol becomes limited. As such, formaldehyde behaves as a light component. In view of these difficulties, as well as others, the separation of streams that comprise water and formaldehyde cannot be expected to behave as a typical two-component system. These features contribute to the unpredictability and difficulty of the separation of the unique crude product stream of the present invention.

The present invention, surprisingly and unexpectedly, achieves effective separation of alkylenating agent(s) from the inventive crude product stream to yield a purified product comprising acrylate product and very low amounts of other impurities.

In one embodiment, the alkylenating split is performed such that a lower amount of acetic acid is present in the resulting alkylenating stream. Preferably, the alkylenating agent stream comprises little or no acetic acid. As an example, the alkylenating agent stream, in some embodiments, comprises less than 50 wt % acetic acid, e.g., less than 45 wt %, less than 25 wt %, less than 10 wt %, less than 5 wt %, less than 3 wt %, or less than 1 wt %. Surprisingly and unexpectedly, the present invention provides for the lower amounts of acetic acid in the alkylenating agent stream, which, beneficially reduces or eliminates the need for further treatment of the alkylenating agent stream to remove acetic acid. In some embodiments, the alkylenating agent stream may be treated to remove water therefrom, e.g., to purge water.

In some embodiments, the alkylenating agent split is performed in at least one column, e.g., at least two columns or at least three columns. Preferably, the alkylenating agent is performed in a two column system. In other embodiments, the alkylenating agent split is performed via contact with an extraction agent. In other embodiments, the alkylenating agent split is performed via precipitation methods, e.g., crystallization, and/or azeotropic distillation. Of course, other suitable separation methods may be employed either alone or in combination with the methods mentioned herein.

In one embodiment, the crude product stream is optionally treated, e.g. separated, prior to the separation of alkylenating agent therefrom. In such cases, the treatment(s) occur before the alkylenating agent split is performed. In other embodiments, at least a portion of the intermediate acrylate product stream may be further treated after the alkylenating agent split. As one example, the crude product stream may be treated to remove light ends therefrom. This treatment may occur either before or after the alkylenating agent split, preferably before the alkylenating agent split. In some of these cases, the further treatment of the intermediate acrylate product stream may result in derivative streams that may be considered to be additional purified acrylate product streams. In other embodiments, the further treatment of the intermediate acrylate product stream results in at least one purified acrylate product stream.

The intermediate product stream comprises acrylate products. In one embodiment, the intermediate product stream comprises a significant portion of acrylate products, e.g., acrylic acid. For example, the intermediate product stream may comprise at least 5 wt % acrylate products, e.g., at least 25 wt %, at least 40 wt %, at least 50 wt %, or at least 60 wt %. In terms of ranges, the intermediate product stream may comprise from 5 wt % to 99 wt % acrylate products, e.g. from 10 wt % to 90 wt %, from 25 wt % to 75 wt %, or from 35 wt % to 65 wt %. The intermediate product stream, in one embodiment, comprises little if any alkylenating agent. For example, the intermediate product stream may comprise less than 1 wt % alkylenating agent, e.g., less than 0.1 wt % alkylenating agent, less than 0.05 wt %, or less than 0.01 wt %. In addition to the acrylate products, the intermediate product stream optionally comprises acetic acid, water, propionic acid and other components.

In some embodiments, the intermediate acrylate product stream further comprises acetic acid. For example in one embodiment, the intermediate acrylate product stream comprises at least 5 wt. % acetic acid, e.g., at least 15 wt %, at least 20 wt. % or at least 40 wt. %. In terms of ranges, the intermediate acrylate product stream may comprise from 10 wt. % to 70 wt. % acetic acid, e.g., from 20 wt. % to 60 wt. %, or from 30 wt. % to 50 wt. %.

To obtain a purified acrylate product stream from the intermediate acrylate product stream, it is desirable to separate the acetic acid therefrom. It has now been found that this intermediate acrylate product stream, when separated via azeotropic distillation using entrainer(s), yields a high purity acrylic acid stream. Preferably, the alkylenating agent split is performed before the separation of acrylic acid and acetic acid, e.g., via azeotropic distillation. The use of the alkylenating agent split and/or azeotropic distillation yields an acrylate product, e.g., acrylic acid, having a higher concentration of acrylate product and a lower concentration of impurities as compared to a conventional separation scheme that employs standard distillation unit(s). Accordingly, in one embodiment of the present invention, the intermediate acrylate product stream is separated via azeotropic distillation using entrainer(s) into an acrylate product stream and an entrainer stream.

The acrylate product stream comprises acrylate product and the entrainer stream comprises entrainer and acetic acid. For example, the acrylate product stream may comprise at least 5 wt. % acrylate product, e.g., acrylic acid, e.g., at least 20 wt. % or at least 35 wt. %. In terms of ranges, the acrylate product stream may comprise from 5 wt. % to 99 wt. % acrylate product, e.g., acrylic acid, e.g., from 20 wt. % to 95 wt. % or from 35 wt. % to 90 wt. %. In one embodiment, the entrainer stream may comprise at least 30 wt. % entrainer, e.g., at least 40 wt. % or at least 50 wt. %. In terms of ranges, the entrainer stream may comprise from 30 wt. % to 99 wt. % entrainer, e.g., from 40 wt. % to 85 wt. % or from 50 wt. % to 80 wt. %. The compositions of the acrylate product stream and the entrainer are further discussed below. In one embodiment, at least a portion of the entrainer in the entrainer stream may be recovered and reused in the separation process.

Without being bound by theory, because of the small relative volatilities of acrylic acid and acetic acid, the separation of acrylic acid and acetic acid, when performed in a distillation column, typically requires: 1) a large number of distillation plates and; 2) a high reflux ratio for the distillation column. The use of large number of distillation plates requires higher temperature in the reboiler and may result in the undesirable polymerization of acrylic acid. The use of azeotropic distillation with entrainer(s) in accordance with the present invention surprisingly and unexpectedly improves the efficiency of the recovery of acrylic acid from the intermediate acrylate product stream, thus reducing both the number of distillation plates that are required and the reflux ratio for the distillation column. As a result, a more efficient separation is achieved and the polymerization of acrylic acid is reduced. In preferred embodiments, the entrainer(s) may be any suitable agent that is capable of increasing the relative volatility between acrylic acid and acetic acid in the intermediate acrylate product stream. Without being bound by theory, it is believed that the entrainer forms an azeotrope with acetic acid and thereby facilitating the recovery of pure acrylic acid. In some embodiments, multiple entrainers, e.g., two or more or three or more, may be used in the distillation column to further increase the relative volatilities of acrylic acid and other components. In one embodiment, the entrainer(s) may be selected from a group consisting of toluene, methyl-isobutyl ketone, di-isobutyl ketone, o-xylene, p-xylene, benzene, n-hexane, cyclohexane, isopropyl-alcohol, diphenyl ether, biphenyl, n-butylacetate, i-butylacetate, derivatives thereof, and combinations thereof.

As noted above, the entrainer stream comprises entrainer, as well as unreacted acetic acid, and alkylenating agent. For example, the entrainer stream may comprise at least 5 wt. % acetic acid, e.g., at least 15 wt. %, at least 20 wt. % or at least 25 wt. %. In terms of ranges, the entrainer stream may comprise from 5 wt. % to 65 wt. % acetic acid, from 15 wt. % to 55 wt. % or from 20 wt. % to 50 wt. %. The entrainer stream may also comprise substantially all of the alkylenating agent in the crude product stream. For example, the entrainer stream may comprise at least 85% of the alkylenating agent, e.g., at least 90%, at least 95%, at least 99% or at least 99.5%. In one embodiment, the entrainer stream may comprise a small amount of acrylate products. For example, the entrainer stream may comprise at most 5 wt. % acrylate product, at most 3 wt. %, at most 1 wt. % or at most 0.8 wt. %. In terms of ranges, the entrainer stream may comprise from 0.001 wt. % to 10 wt. % acrylate product, from 0.5 wt. % to 5 wt. %, from 0.8 wt. % to 5 wt. %.

In an embodiment, the acrylate product stream comprises acrylate products, e.g., acrylic acid. In one embodiment, the acrylate product stream comprises a significant portion of acrylic acid, as discussed above. The acrylate product stream, in one embodiment, comprises little if any alkylenating agent. For example, the acrylate product stream may comprise less than 1 wt. % alkylenating agent, e.g., less than 0.1 wt. % alkylenating agent, less than 0.05 wt. %, or less than 0.01 wt. %. In addition to the acrylate products, the acrylate product stream optionally comprises acetic acid, water, propionic acid and other components.

In one embodiment, the acrylate product stream may be further purified in a distillation column, i.e., “product recovery column,” to yield a finished acrylate product stream as a residue stream. The finished acrylate product stream comprises mostly acrylate product, i.e., acrylic acid. In an embodiment, the finished acrylate product stream comprises at least 60 wt. % acrylic acid, e.g., at least 70 wt. %, at least 80 wt. % or at least 85 wt. %. In terms of ranges, the finished acrylate product stream comprises from 60 wt. % to 99 wt. % acrylic acid, e.g., from 70 wt. % to 97 wt. %, from 80 wt. % to 95 wt. %, or from 82 wt. % to 90 wt. %. The finished acrylate product stream may comprise a small amount of acetic acid. For example, the finished acrylate product stream may comprise less than 30 wt. % acetic acid, e.g., less than 15 wt. %, less than 12 wt. % or less than 10 wt. %. The finished acrylate product stream may also comprise a small amount of entrainer. For example, the finished acrylate product stream may comprise less than 45 wt. % entrainer, e.g., less than 35 wt. %, less than 25 wt. % or less than 15 wt. %. In one embodiment, the finished acrylate product stream may be further purified to recover an ultra high purity acrylic acid product, i.e., 99 wt. % acrylic acid. In one embodiment, the finished acrylate product stream comprises at least 60 wt. % acrylic acid, e.g., at least 70 wt. %, at least 80 wt. %, or at least 85 wt. %.

In an embodiment, the distillate stream of the product recovery column may comprise acrylic acid, acetic acid and entrainer. For example, the distillate stream may comprise at least 5 wt. % acrylic acid, e.g., at least 10 wt. %, at least 20 wt. %, or at least 40 wt. %. In terms of ranges, the distillate stream may comprise from 5 wt. % to 80 wt. % acrylic acid, e.g., from 10 wt. % to 70 wt. %, from 20 wt. % to 60 wt. % or from 40 wt. % to 50 wt. %. The distillate stream may comprise at least 5 wt. % acetic acid, e.g., at least 10 wt. %, at least 20 wt. %, or at least 30 wt. %. In terms of ranges, the distillate stream may comprise from 5 wt. % to 80 wt. % acetic acid, e.g., from 10 wt. % to 70 wt. %, from 20 wt. % to 60 wt. % or from 30 wt. % to 50 wt. %. The distillate stream may comprise at least 1 wt. % entrainer, e.g., at least 5 wt. %, at least 10 wt. %, at least 15 wt. %, or at least 20 wt. %. In terms of ranges, the distillate stream may comprise from 1 wt. % to 50 wt. % entrainer, e.g., from 5 wt. % to 40 wt. %, from 10 wt. % to 35 wt. %, from 15 wt. % to 25 wt. % or from 20 wt. % to 23 wt. %. In an embodiment, the entrainer in the distillate stream may be recovered and recycled to the azeotropic distillation column.

In one embodiment, the inventive process beneficially reduces or eliminates the need for further treatment of the acrylate product stream to remove unreacted acetic acid therefrom. In some embodiments however, entrainer(s) may be separated from the acrylic product stream. In some embodiments, the separation of the unreacted acetic acid and the acrylate products is performed in at least one column, e.g., at least two columns or at least three columns. Preferably, the acrylic acid/unreacted acetic acid separation is performed in a one column system. In one embodiment, unreacted acetic acid is recovered from the entrainer stream and may be recycled to the reactor directly or indirectly.

The use of an azeotropic distillation column in accordance with embodiments of the present invention allows for a majority of the acrylic acid to be recovered from the intermediate acrylate product stream. Preferably, at least 90% of the acrylic acid in the intermediate acrylate product stream is recovered in the residue stream of the azeotropic distillation column, e.g., at least 95% or at least 98%. Recovering a majority of the acrylic acid in the residue provides for low concentrations of unrecovered acrylic acid in the distillate, e.g., less than 2 wt. %, less than 0.5 wt. % or less than 0.01 wt. %. Thus, additional separations to recover acrylic acid are reduced or eliminated. Furthermore, the low concentration of unrecovered acrylic in the distillate stream reduces the potential for polymerization of the unrecovered acrylic acid when the stream is vaporized prior to being recycled to the reactor.

In one embodiment, entrainer(s) may be recovered by using a distillation column from the entrainer stream and/or the distillate stream of the product recovery column. As stated above, both the entrainer stream and the distillate stream of the product recovery column contains entrainers. In some embodiments, entrainer may be recovered from the two streams separately. In some embodiments, the two streams may be combined together to form an entrainer-containing stream and entrainer agent may be recovered therefrom. In another embodiment, the entrainers may be recovered using a combination of distillation columns and extraction units. For example, the azeotropic distillation column may be coupled with a liquid-liquid extraction unit for the recovery of entrainers.

In one embodiment, the entrainer-containing stream may be separated into an entrainer recovery stream and an acetic acid stream. The entrainer recovery stream comprises a significant portion of entrainer. For example, the entrainer recovery stream may comprise at least 15 wt. % entrainer, e.g., at least 30 wt. %, at least 50 wt. %, at least 65 wt. % or at least 80 wt. %. In terms of ranges, the entrainer recovery stream may comprise from 30 wt. % to 99 wt. % entrainer, e.g. from 50 wt. % to 97 wt. %, from 60 wt. % to 95 wt. %, or from 80 wt. % to 90 wt. %. The entrainer recovery stream may comprise some acetic acid. For example, the entrainer recovery stream may comprise less than 50 wt. % acetic acid, e.g., less than 30 wt. %, less than 20 wt. %, or less than 10 wt. %. The entrainer recovery stream may comprise substantially all the alkylenating agent in the entrainer stream from the azeotropic column. For example, the entrainer stream may comprise at least 85% of the alkylenating agent, e.g., at least 90%, at least 95%, at least 99% or at least 99.5%. In one embodiment, the entrainer recovery stream is returned to the azeotropic distillation column.

In one embodiment, an entrainer recovery column yields an acetic acid stream. The acetic acid stream comprises acetic acid, entrainer and a small amount, if any, of acrylate product. For example, the acetic acid stream comprises at least 10 wt. % acetic acid, e.g., at least 30 wt. %, at least 40 wt. %, or at least 50 wt. %. In terms of ranges, the acetic acid stream may comprise from 10 wt. % to 95 wt. % acetic acid, e.g. from 30 wt. % to 90 wt. %, from 40 wt. % to 80 wt. %, or from 50 wt. % to 70 wt. %. The acetic acid stream may also comprise entrainer agent. For example, the acetic acid stream may comprise at most 60 wt. % entrainer agent, e.g., at most 50 wt. %, at most 40 wt. % or at most 35 wt. %. The acetic acid stream may also comprise a small amount of acrylic acid. For example, the acetic acid stream may comprise at most 30 wt. % acrylic acid, e.g., at most 20 wt. %, at most 15 wt. % or at most 10 wt. %. In one embodiment, the acetic acid stream is returned, directly or indirectly, to the reactor.

Using an entrainer in accordance with embodiments of the present invention allows for a majority of the acrylic acid to be recovered from the intermediate acrylate product stream. Preferably, In one embodiment, the inventive process operates at a high process efficiency. For example, the process efficiency may be at least 10%, e.g., at least 20% or at least 35%. In one embodiment, the process efficiency is calculated based on the flows of reactants into the reaction zone. The process efficiency may be calculated by the following formula.

Process Efficiency=2N_{HAcA}/[N_{HCHO}+N_{HCHO}+N_{H2O}]

where:

N_{HAcA} is the molar production rate of acrylate products; and

N_{HOAc}, N_{HCHO}, and N_{H2O} are the molar feed rates of acetic acid, formaldehyde, and water.

### Production of Acrylate Products

Any suitable reaction and/or separation scheme may be employed to form the crude product stream as long as the reaction provides the crude product stream components that are discussed above. For example, in some embodiments, the acrylate product stream is formed by contacting an alkanoic acid, e.g., acetic acid, or an ester thereof with an alkylenating agent, e.g., a methylenating agent, for example formaldehyde, under conditions effective to form the crude acrylate product stream. Preferably, the contacting is performed over a suitable catalyst. The crude product stream may be the reaction product of the alkanoic acid-alkylenating agent reaction. In a preferred embodiment, the crude product stream is the reaction product of the aldol condensation reaction of acetic acid and formaldehyde, which is conducted over a catalyst comprising vanadium and titanium. In one embodiment, the crude product stream is the product of a reaction in wherein methanol and acetic acid are combined to generate formaldehyde in situ. The aldol condensation then follows. In one embodiment, a methanol-formaldehyde solution is reacted with acetic acid to form the crude product stream.

The alkanoic acid, or an ester of the alkanoic acid, may be of the formula R′—CH₂—COOR, where R and R′ are each, independently, hydrogen or a saturated or unsaturated alkyl or aryl group. As an example, R and R′ may be a lower alkyl group containing for example 1-4 carbon atoms. In one embodiment, an alkanoic acid anhydride may be used as the source of the alkanoic acid. In one embodiment, the reaction is conducted in the presence of an alcohol, preferably the alcohol that corresponds to the desired ester, e.g., methanol. In addition to reactions used in the production of acrylic acid, the inventive catalyst, in other embodiments, may be employed to catalyze other reactions.

The alkanoic acid, e.g., acetic acid, may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. As petroleum and natural gas prices fluctuate, becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syngas”) that is derived from any available carbon source. U.S. Pat. No. 6,232,352, which is hereby incorporated by reference, for example, teaches a method of retrofitting a methanol plant for the manufacture of acetic acid. By retrofitting a methanol plant, the large capital costs associated with carbon monoxide generation for a new acetic acid plant are significantly reduced or largely eliminated. All or part of the syngas is diverted from the methanol synthesis loop and supplied to a separator unit to recover carbon monoxide and hydrogen, which are then used to produce acetic acid.

In some embodiments, at least some of the raw materials for the above-described aldol condensation process may be derived partially or entirely from syngas. For example, the acetic acid may be formed from methanol and carbon monoxide, both of which may be derived from syngas. For example, the methanol may be formed by steam reforming syngas, and the carbon monoxide may be separated from syngas. In other embodiments, the methanol may be formed in a carbon monoxide unit, e.g., as described in EP2076480; EP1923380; EP2072490; EP1914219; EP1904426; EP2072487; E02072492; EP2072486; EP2060553; EP1741692; EP1907344; EP2060555; EP2186787; EP2072488; and U.S. Pat. No. 7,842,844. Of course, this listing of methanol sources is merely exemplary and is not meant to be limiting. In addition, the above-identified methanol sources, inter alia, may be used to form the formaldehyde, e.g., in situ, which, in turn may be reacted with the acetic acid to form the acrylic acid. The syngas, in turn, may be derived from variety of carbon sources. The carbon source, for example, may be selected from the group consisting of natural gas, oil, petroleum, coal, biomass, and combinations thereof.

Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259 and 4,994,608, all of which are hereby incorporated by reference.

U.S. Pat. No. RE 35,377, which is hereby incorporated by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. U.S. Pat. No. 5,821,111, which discloses a process for converting waste biomass through gasification into syngas, as well as U.S. Pat. No. 6,685,754 are hereby incorporated by reference.

In one optional embodiment, the acetic acid that is utilized in the condensation reaction comprises acetic acid and may also comprise other carboxylic acids, e.g., propionic acid, esters, and anhydrides, as well as acetaldehyde and acetone. In one embodiment, the acetic acid fed to the condensation reaction comprises propionic acid. For example, the acetic acid fed to the reaction may comprise from 0.001 wt. % to 15 wt. % propionic acid, e.g., from 0.125 wt. % to 12.5 wt. %, from 1.25 wt. % to 11.25 wt. %, or from 3.75 wt. % to 8.75 wt. %. Thus, the acetic acid feed stream may be a cruder acetic acid feed stream, e.g., a less-refined acetic acid feed stream.

As used herein, “alkylenating agent” means an aldehyde or precursor to an aldehyde suitable for reacting with the alkanoic acid, e.g., acetic acid, to form an unsaturated acid, e.g., acrylic acid, or an alkyl acrylate. In preferred embodiments, the alkylenating agent comprises a methylenating agent such as formaldehyde, which preferably is capable of adding a methylene group (═CH₂) to the organic acid. Other alkylenating agents may include, for example, acetaldehyde, propanal, butanal, aryl aldehydes, benzyl aldehydes, alcohols, and combinations thereof. This listing is not exclusive and is not meant to limit the scope of the invention. In one embodiment, an alcohol may serve as a source of the alkylenating agent. For example, the alcohol may be reacted in situ to form the alkylenating agent, e.g., the aldehyde.

The alkylenating agent, e.g., formaldehyde, may be derived from any suitable source. Exemplary sources may include, for example, aqueous formaldehyde solutions, anhydrous formaldehyde derived from a formaldehyde drying procedure, trioxane, diether of methylene glycol, and paraformaldehyde. In a preferred embodiment, the formaldehyde is produced via a methanol oxidation process, which reacts methanol and oxygen to yield the formaldehyde.

In other embodiments, the alkylenating agent is a compound that is a source of formaldehyde. Where forms of formaldehyde that are not as freely or weakly complexed are used, the formaldehyde will form in situ in the condensation reactor or in a separate reactor prior to the condensation reactor. Thus for example, trioxane may be decomposed over an inert material or in an empty tube at temperatures over 350° C. or over an acid catalyst at over 100° C. to form the formaldehyde.

In one embodiment, the alkylenating agent corresponds to Formula I.

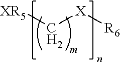

In this formula, R₅ and R₆ may be independently selected from C₁-C₁₂ hydrocarbons, preferably, C₁-C₁₂ alkyl, alkenyl or aryl, or hydrogen. Preferably, R₅ and R₆ are independently C₁-C₆ alkyl or hydrogen, with methyl and/or hydrogen being most preferred. X may be either oxygen or sulfur, preferably oxygen; and n is an integer from 1 to 10, preferably 1 to 3. In some embodiments, m is 1 or 2, preferably 1.

In one embodiment, the compound of formula I may be the product of an equilibrium reaction between formaldehyde and methanol in the presence of water. In such a case, the compound of formula I may be a suitable formaldehyde source. In one embodiment, the formaldehyde source includes any equilibrium composition. Examples of formaldehyde sources include but are not restricted to methylal (1,1 dimethoxymethane); polyoxymethylenes —(CH₂—O)ᵢ— wherein i is from 1 to 100; formalin; and other equilibrium compositions such as a mixture of formaldehyde, methanol, and methyl propionate. In one embodiment, the source of formaldehyde is selected from the group consisting of 1,1 dimethoxymethane; higher formals of formaldehyde and methanol; and CH₃—O—(CH₂—O)ᵢ—CH₃ where i is 2.

The alkylenating agent may be used with or without an organic or inorganic solvent.

The term “formalin,” refers to a mixture of formaldehyde, methanol, and water. In one embodiment, formalin comprises from 25 wt. % to 65 wt. % formaldehyde; from 0.01 wt. % to 25 wt. % methanol; and from 25 wt. % to 70 wt. % water. In cases where a mixture of formaldehyde, methanol, and methyl propionate is used, the mixture comprises less than 10 wt. % water, e.g., less than 5 wt. % or less than 1 wt. %.

In some embodiments, the condensation reaction may achieve favorable conversion of acetic acid and favorable selectivity and productivity to acrylates. For purposes of the present invention, the term “conversion” refers to the amount of acetic acid in the feed that is converted to a compound other than acetic acid. Conversion is expressed as a percentage based on acetic acid in the feed. The conversion of acetic acid may be at least 10%, e.g., at least 20%, at least 40%, or at least 50%.

Selectivity, as it refers to the formation of acrylate product, is expressed as the ratio of the amount of carbon in the desired product(s) and the amount of carbon in the total products. This ratio may be multiplied by 100 to arrive at the selectivity. Preferably, the catalyst selectivity to acrylate products, e.g., acrylic acid and methyl acrylate, is at least 40 mol %, e.g., at least 50 mol %, at least 60 mol %, or at least 70 mol %. In some embodiments, the selectivity to acrylic acid is at least 30 mol %, e.g., at least 40 mol %, or at least 50 mol %; and/or the selectivity to methyl acrylate is at least 10 mol %, e.g., at least 15 mol %, or at least 20 mol %.

The terms “productivity” or “space time yield” as used herein, refers to the grams of a specified product, e.g., acrylate products, formed per hour during the condensation based on the liters of catalyst used. A productivity of at least 20 grams of acrylate product per liter catalyst per hour, e.g., at least 40 grams of acrylates per liter catalyst per hour or at least 100 grams of acrylates per liter catalyst per hour, is preferred. In terms of ranges, the productivity preferably is from 20 to 500 grams of acrylates per liter catalyst per hour, e.g., from 20 to 200 grams of acrylates per liter catalyst per hour or from 40 to 140 grams of acrylates per liter catalyst per hour.

In one embodiment, the inventive process yields at least 1,800 kg/hr of finished acrylic acid, e.g., at least 3,500 kg/hr, at least 18,000 kg/hr, or at least 37,000 kg/hr.

Preferred embodiments of the inventive process demonstrate a low selectivity to undesirable products, such as carbon monoxide and carbon dioxide. The selectivity to these undesirable products preferably is less than 29%, e.g., less than 25% or less than 15%. More preferably, these undesirable products are not detectable. Formation of alkanes, e.g., ethane, may be low, and ideally less than 2%, less than 1%, or less than 0.5% of the acetic acid passed over the catalyst is converted to alkanes, which have little value other than as fuel.

The alkanoic acid or ester thereof and alkylenating agent may be fed independently or after prior mixing to a reactor containing the catalyst. The reactor may be any suitable reactor or combination of reactors. Preferably, the reactor comprises a fixed bed reactor or a series of fixed bed reactors. In one embodiment, the reactor is a packed bed reactor or a series of packed bed reactors. In one embodiment, the reactor is a fixed bed reactor. Of course, other reactors such as a continuous stirred tank reactor or a fluidized bed reactor, may be employed.

In some embodiments, the alkanoic acid, e.g., acetic acid, and the alkylenating agent, e.g., formaldehyde, are fed to the reactor at a molar ratio of at least 0.10:1, e.g., at least 0.75:1 or at least 1:1. In terms of ranges the molar ratio of alkanoic acid to alkylenating agent may range from 0.10:1 to 10:1 or from 0.75:1 to 5:1. In some embodiments, the reaction of the alkanoic acid and the alkylenating agent is conducted with a stoichiometric excess of alkanoic acid. In these instances, acrylate selectivity may be improved. As an example the acrylate selectivity may be at least 10% higher than a selectivity achieved when the reaction is conducted with an excess of alkylenating agent, e.g., at least 20% higher or at least 30% higher. In other embodiments, the reaction of the alkanoic acid and the alkylenating agent is conducted with a stoichiometric excess of alkylenating agent.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. Residence time in the reactor may range from 1 second to 200 seconds, e.g., from 1 second to 100 seconds. Reaction pressure is not particularly limited, and the reaction is typically performed near atmospheric pressure. In one embodiment, the reaction may be conducted at a pressure ranging from 0 kPa to 4100 kPa, e.g., from 3 kPa to 345 kPa, or from 6 kPa to 103 kPa. The acetic acid conversion, in some embodiments, may vary depending upon the reaction temperature.

In one embodiment, the reaction is conducted at a gas hourly space velocity (“GHSV”) greater than 600 hr⁻¹, e.g., greater than 1000 hr⁻¹ or greater than 2000 hr⁻¹. In one embodiment, the GHSV ranges from 600 hr⁻¹ to 10000 hr⁻¹, e.g., from 1000 hr⁻¹ to 8000 hr⁻¹ or from 1500 hr⁻¹ to 7500 hr⁻¹. As one particular example, when GHSV is at least 2000 hr⁻¹, the acrylate product STY may be at least 150 g/hr/liter.

Water may be present in the reactor in amounts up to 60 wt. %, by weight of the reaction mixture, e.g., up to 50 wt. % or up to 40 wt. %. Water, however, is preferably reduced due to its negative effect on process rates and separation costs.

In one embodiment, an inert or reactive gas is supplied to the reactant stream. Examples of inert gases include, but are not limited to, nitrogen, helium, argon, and methane. Examples of reactive gases or vapors include, but are not limited to, oxygen, carbon oxides, sulfur oxides, and alkyl halides. When reactive gases such as oxygen are added to the reactor, these gases, in some embodiments, may be added in stages throughout the catalyst bed at desired levels as well as feeding with the other feed components at the beginning of the reactors. The addition of these additional components may improve reaction efficiencies.

In one embodiment, the unreacted components such as the alkanoic acid and formaldehyde as well as the inert or reactive gases that remain are recycled to the reactor after sufficient separation from the desired product.

When the desired product is an unsaturated ester made by reacting an ester of an alkanoic acid ester with formaldehyde, the alcohol corresponding to the ester may also be fed to the reactor either with or separately to the other components. For example, when methyl acrylate is desired, methanol may be fed to the reactor. The alcohol, amongst other effects, reduces the quantity of acids leaving the reactor. It is not necessary that the alcohol is added at the beginning of the reactor and it may for instance be added in the middle or near the back, in order to effect the conversion of acids such as propionic acid, methacrylic acid to their respective esters without depressing catalyst activity. In one embodiment, the alcohol may be added downstream of the reactor.

### Catalyst Composition

The catalyst may be any suitable catalyst composition. As one example, condensation catalyst consisting of mixed oxides of vanadium and phosphorus have been investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). Other examples include binary vanadium-titanium phosphates, vanadium-silica-phosphates, and alkali metal-promoted silicas, e.g., cesium- or potassium-promoted silicas.

In a preferred embodiment, the inventive process employs a catalyst composition comprising vanadium, titanium, and optionally at least one oxide additive. The oxide additive(s), if present, are preferably present in the active phase of the catalyst. In one embodiment, the oxide additive(s) are selected from the group consisting of silica, alumina, zirconia, and mixtures thereof or any other metal oxide other than metal oxides of titanium or vanadium. Preferably, the molar ratio of oxide additive to titanium in the active phase of the catalyst composition is greater than 0.05:1, e.g., greater than 0.1:1, greater than 0.5:1, or greater than 1:1. In terms of ranges, the molar ratio of oxide additive to titanium in the inventive catalyst may range from 0.05:1 to 20:1, e.g., from 0.1:1 to 10:1, or from 1:1 to 10:1. In these embodiments, the catalyst comprises titanium, vanadium, and one or more oxide additives and have relatively high molar ratios of oxide additive to titanium.

In other embodiments, the catalyst may further comprise other compounds or elements (metals and/or non-metals). For example, the catalyst may further comprise phosphorus and/or oxygen. In these cases, the catalyst may comprise from 15 wt. % to 45 wt. % phosphorus, e.g., from 20 wt. % to 35 wt. % or from 23 wt. % to 27 wt. %; and/or from 30 wt. % to 75 wt. % oxygen, e.g., from 35 wt. % to 65 wt. % or from 48 wt. % to 51 wt. %.

In some embodiments, the catalyst further comprises additional metals and/or oxide additives. These additional metals and/or oxide additives may function as promoters. If present, the additional metals and/or oxide additives may be selected from the group consisting of copper, molybdenum, tungsten, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference.

If the catalyst comprises additional metal(s) and/or metal oxides(s), the catalyst optionally may comprise additional metals and/or metal oxides in an amount from 0.001 wt. % to 30 wt. %, e.g., from 0.01 wt. % to 5 wt. % or from 0.1 wt. % to 5 wt. %. If present, the promoters may enable the catalyst to have a weight/weight space time yield of at least 25 grams of acrylic acid/gram catalyst-h, e.g., least 50 grams of acrylic acid/gram catalyst-h, or at least 100 grams of acrylic acid/gram catalyst-h.

In some embodiments, the catalyst is unsupported. In these cases, the catalyst may comprise a homogeneous mixture or a heterogeneous mixture as described above. In one embodiment, the homogeneous mixture is the product of an intimate mixture of vanadium and titanium oxides, hydroxides, and phosphates resulting from preparative methods such as controlled hydrolysis of metal alkoxides or metal complexes. In other embodiments, the heterogeneous mixture is the product of a physical mixture of the vanadium and titanium phosphates. These mixtures may include formulations prepared from phosphorylating a physical mixture of preformed hydrous metal oxides. In other cases, the mixture(s) may include a mixture of preformed vanadium pyrophosphate and titanium pyrophosphate powders.

In another embodiment, the catalyst is a supported catalyst comprising a catalyst support in addition to the vanadium, titanium, oxide additive, and optionally phosphorous and oxygen, in the amounts indicated above (wherein the molar ranges indicated are without regard to the moles of catalyst support, including any vanadium, titanium, oxide additive, phosphorous or oxygen contained in the catalyst support). The total weight of the support (or modified support), based on the total weight of the catalyst, preferably is from 75 wt. % to 99.9 wt. %, e.g., from 78 wt. % to 97 wt. % or from 80 wt. % to 95 wt. %. The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. In embodiments where the catalyst comprises a titania support, the titania support may comprise a major or minor amount of rutile and/or anatase titanium dioxide. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, other microporous and mesoporous materials, and mixtures thereof. Other supports may include, but are not limited to, iron oxide, magnesia, steatite, magnesium oxide, carbon, graphite, high surface area graphitized carbon, activated carbons, and mixtures thereof. These listings of supports are merely exemplary and are not meant to limit the scope of the present invention.

In some embodiments, a zeolitic support is employed. For example, the zeolitic support may be selected from the group consisting of montmorillonite, NH₄ ferrierite, H-mordenite-PVOx, vermiculite-1, H-ZSM5, NaY, H-SDUSY, Y zeolite with high SAR, activated bentonite, H-USY, MONT-2, HY, mordenite SAR 20, SAPO-34, Aluminosilicate (X), VUSY, Aluminosilicate (CaX), Re-Y, and mixtures thereof. H-SDUSY, VUSY, and H-USY are modified Y zeolites belonging to the faujasite family. In one embodiment, the support is a zeolite that does not contain any metal oxide modifier(s). In some embodiments, the catalyst composition comprises a zeolitic support and the active phase comprises a metal selected from the group consisting of vanadium, aluminum, nickel, molybdenum, cobalt, iron, tungsten, zinc, copper, titanium cesium bismuth, sodium, calcium, chromium, cadmium, zirconium, and mixtures thereof. In some of these embodiments, the active phase may also comprise hydrogen, oxygen, and/or phosphorus.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals and/or pyrophosphates that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

### Separation

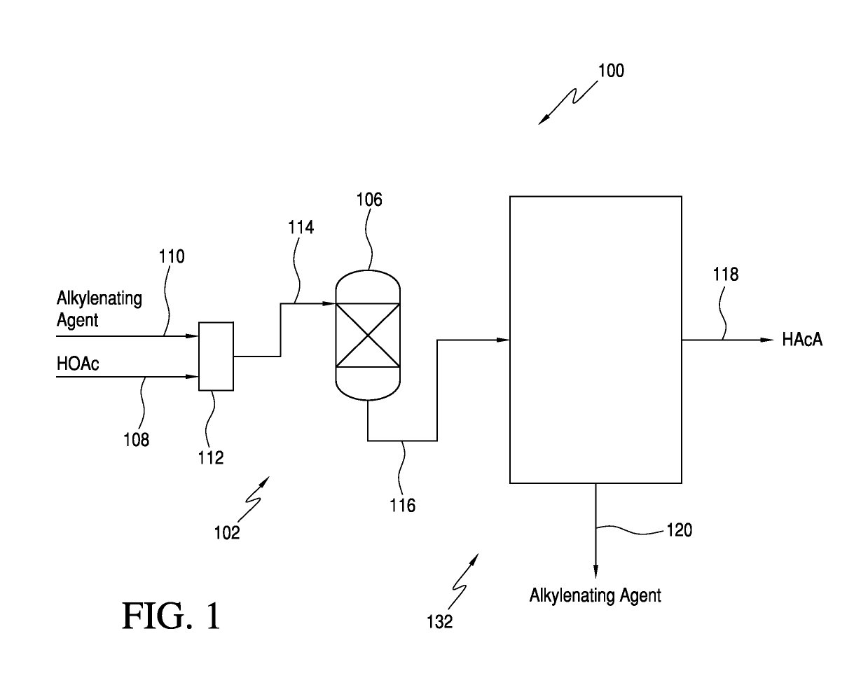

As discussed above, the crude product stream is separated to yield an intermediate acrylate product stream. FIG. 1 is a flow diagram depicting the formation of the crude product stream and the separation thereof to obtain an intermediate acrylate product stream. Acrylate product system 100 comprises reaction zone 102 and alkylenating agent split zone 132. Reaction zone 102 comprises reactor 106, alkanoic acid feed, e.g., acetic acid feed, 108, alkylenating agent feed, e.g., formaldehyde feed 110, and vaporizer 112.

Acetic acid and formaldehyde are fed to vaporizer 112 via lines 108 and 110, respectively, to create a vapor feed stream, which exits vaporizer 112 via line 114 and is directed to reactor 106.

In one embodiment, lines 108 and 110 may be combined and jointly fed to the vaporizer 112. The temperature of the vapor feed stream in line 114 is preferably from 200° C. to 600° C., e.g., from 250° C. to 500° C. or from 340° C. to 425° C. Alternatively, a vaporizer may not be employed and the reactants may be fed directly to reactor 106.

Any feed that is not vaporized may be removed from vaporizer 112 and may be recycled or discarded. In addition, although line 114 is shown as being directed to the upper half of reactor 106, line 114 may be directed to the middle or bottom of first reactor 106. Further modifications and additional components to reaction zone 102 and alkylenating agent split zone 132 are described below.

Reactor 106 contains the catalyst that is used in the reaction to form crude product stream, which is withdrawn, preferably continuously, from reactor 106 via line 116. Although FIG. 1 shows the crude product stream being withdrawn from the bottom of reactor 106, the crude product stream may be withdrawn from any portion of reactor 106. Exemplary composition ranges for the crude product stream are shown in Table 1 above.

In one embodiment, one or more guard beds (not shown) may be used upstream of the reactor to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens.

The crude product stream in line 116 is fed to alkylenating agent split unit 132. Alkylenating agent split unit 132 may comprise one or more separation units, e.g., two or more or three or more. In one example, the alkylenating agent split unit contains multiple columns, as shown in FIG. 1. Alkylenating agent split unit 132 separates the crude product stream into at least one intermediate acrylate product stream, which exits via line 118 and at least one alkylenating agent stream, which exits via line 120. Exemplary compositional ranges for the intermediate acrylate product stream are shown in Table 2. Components other than those listed in Table 2 may also be present in the intermediate acrylate product stream. Examples include methanol, methyl acetate, methyl acrylate, dimethyl ketone, carbon dioxide, carbon monoxide, oxygen, nitrogen, and acetone.

TABLE 2
INTERMEDIATE ACRYLATE PRODUCT
STREAM COMPOSITION

| | Conc. (wt %) | Conc. (wt %) | Conc. (wt %) |
|---|---|---|---|
| Acrylic Acid | at least 5 | 5 to 99 | 35 to 65 |
| Acetic Acid | less than 95 | 5 to 90 | 20 to 60 |
| Water | less than 25 | 0.1 to 10 | 0.5 to 7 |
| Alkylenating Agent | <1 | <0.5 | <0.1 |
| Propionic Acid | <10 | 0.01 to 5 | 0.01 to 1 |

In other embodiments, the intermediate acrylate product stream comprises higher amounts of alkylenating agent. For example, the intermediate acrylate product stream may comprise from 1 wt % to 10 wt % alkylenating agent, e.g., from 1 wt % to 8 wt % or from 2 wt % to 5 wt %. In one embodiment, the intermediate acrylate product stream comprises greater than 1 wt % alkylenating agent, e.g., greater than 5 wt % or greater than 10 wt %.

Exemplary compositional ranges for the alkylenating agent stream are shown in Table 3. Components other than those listed in Table 3 may also be present in the purified alkylate product stream. Examples include methanol, methyl acetate, methyl acrylate, dimethyl ketone, carbon dioxide, carbon monoxide, oxygen, nitrogen, and acetone.

TABLE 3
ALKYLENATING AGENT STREAM COMPOSITION

| | Conc. (wt %) | Conc. (wt %) | Conc. (wt %) |
|---|---|---|---|
| Acrylic Acid | less than 15 | 0.01 to 10 | 0.1 to 5 |
| Acetic Acid | 10 to 65 | 20 to 65 | 25 to 55 |
| Water | 15 to 75 | 25 to 65 | 30 to 60 |
| Alkylenating Agent | at least 1 | 1 to 75 | 10 to 20 |
| Propionic Acid | <10 | 0.001 to 5 | 0.001 to 1 |

In other embodiments, the alkylenating stream comprises lower amounts of acetic acid. For example, the alkylenating agent stream may comprise less than 10 wt % acetic acid, e.g., less than 5 wt % or less than 1 wt %.

As mentioned above, the crude product stream of the present invention comprises little, if any, furfural and/or acrolein. As such the derivative stream(s) of the crude product streams will comprise little, if any, furfural and/or acrolein. In one embodiment, the derivative stream(s), e.g., the streams of the separation zone, comprises less than less than 500 wppm acrolein, e.g., less than 100 wppm, less than 50 wppm, or less than 10 wppm. In one embodiment, the derivative stream(s) comprises less than less than 500 wppm furfural, e.g., less than 100 wppm, less than 50 wppm, or less than 10 wppm.

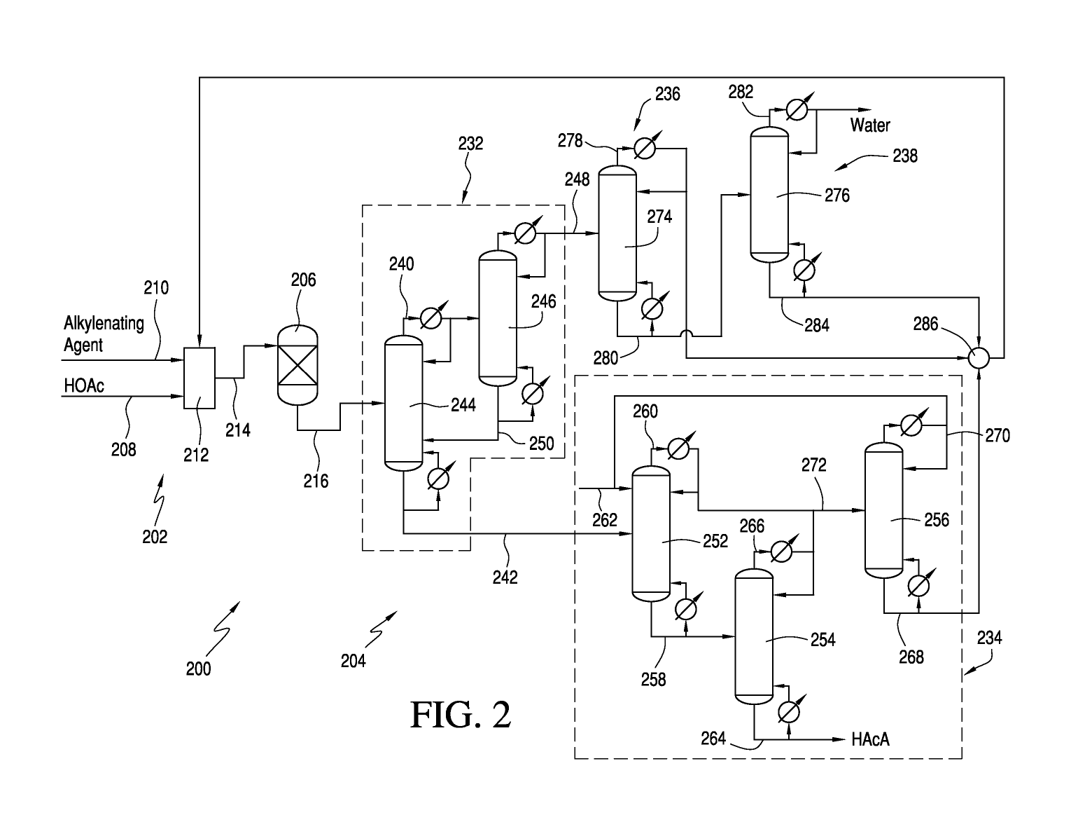

FIG. 2 shows an overview of a reaction/separation scheme in accordance with the present invention. Acrylate product system 200 comprises reaction zone 202 and separation zone 204. Reaction zone 202 comprises reactor 206, alkanoic acid feed, e.g., acetic acid feed, 208, alkylenating agent feed, e.g., formaldehyde feed, 210, vaporizer 212, and line 214. Reaction zone 202 and the components thereof function in a manner similar to reaction zone 102 of FIG. 1.

Reaction zone 202 yields a crude product stream, which exits reaction zone 202 via line 216 and is directed to separation zone 204. The components of the crude product stream are discussed above. Separation zone 204 comprises alkylenating agent split unit 232, acrylate product split unit 234, acetic acid split unit 236, and drying unit 238. Separation zone 204 may also comprise an optional light ends removal unit (not shown). For example, the light ends removal unit may comprise a condenser and/or a flasher. The light ends removal unit may be configured either upstream or downstream of the alkylenating agent split unit. Depending on the configuration, the light ends removal unit removes light ends from the crude product stream, the alkylenating stream, and/or the intermediate acrylate product stream. In one embodiment, when the light ends are removed, the remaining liquid phase comprises the acrylic acid, acetic acid, alkylenating agent, and/or water.

Alkylenating agent split unit 232 may comprise any suitable separation device or combination of separation devices. For example, alkylenating agent split unit 232 may comprise a column, e.g., a standard distillation column, an extractive distillation column and/or an azeotropic distillation column. In other embodiments, alkylenating agent split unit 232 comprises a precipitation unit, e.g., a crystallizer and/or a chiller. Preferably, alkylenating agent split unit 232 comprises two standard distillation columns. In another embodiment, the alkylenating agent split is performed by contacting the crude product stream with a solvent that is immiscible with water. For example alkylenating agent split unit 232 may comprise at least one liquid-liquid extraction columns. In another embodiment, the alkylenating agent split is performed via azeotropic distillation, which employs an azeotropic agent. In these cases, the azeotropic agent may be selected from the group consisting of methyl isobutylketene, o-xylene, toluene, benzene, n-hexane, cyclohexane, p-xylene, and mixtures thereof. This listing is not exclusive and is not meant to limit the scope of the invention. In another embodiment, the alkylenating agent split is performed via a combination of distillation, e.g., standard distillation, and crystallization. Of course, other suitable separation devices may be employed either alone or in combination with the devices mentioned herein.

In FIG. 2, alkylenating agent split unit 232 comprises first column 244 and second column 246. Alkylenating agent split unit 232 receives crude acrylate product stream in line 216 and separates same into at least one alkylenating agent stream, e.g., stream 248, and at least one intermediate acrylate product stream, e.g., stream 242. Alkylenating agent split unit 232 performs an alkylenating agent split, as discussed above.

In operation, as shown in FIG. 2, the crude product stream in line 216 is directed to first column 244. First column 244 separates the crude product stream into a distillate in line 240 and a residue in line 242. The distillate may be refluxed and the residue may be boiled up as shown. Stream 240 comprises at least 1 wt % alkylenating agent. As such, stream 240 may be considered an alkylenating agent stream. The first column residue exits first column 244 in line 242 and comprises a significant portion of acrylate product. As such, stream 242 is an intermediate acrylate product stream. Exemplary compositional ranges for the distillate and residue of first column 244 are shown in Table 4. Components other than those listed in Table 4 may also be present in the residue and distillate.

TABLE 4
FIRST COLUMN (244)

| | Conc. (wt. %) | Conc. (wt. %) | Conc. (wt. %) |
|---|---|---|---|
| Distillate | | | |
| Acrylic Acid | 0.1 to 20 | 1 to 10 | 1 to 5 |
| Acetic Acid | 25 to 65 | 35 to 55 | 40 to 50 |
| Water | 15 to 55 | 25 to 45 | 30 to 40 |
| Alkylenating Agent | at least 1 | 1 to 75 | 10 to 20 |
| Propionic Acid | <10 | 0.001 to 5 | 0.001 to 1 |
| Residue | | | |
| Acrylic Acid | at least 5 | 5 to 99 | 35 to 65 |
| Acetic Acid | less than 95 | 5 to 90 | 20 to 60 |
| Water | less than 25 | 0.1 to 10 | 0.5 to 7 |
| Alkylenating Agent | <1 | <0.5 | <0.1 |
| Propionic Acid | <10 | 0.01 to 5 | 0.01 to 1 |

In one embodiments, the first distillate comprises smaller amounts of acetic acid, e.g., less than 25 wt %, less than 10 wt %, e.g., less than 5 wt % or less than 1 wt %. In other embodiments, the intermediate acrylate product stream comprises higher amounts of alkylenating agent, e.g., greater than 1 wt % greater than 5 wt % or greater than 10 wt %.

For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

In one embodiment, polymerization inhibitors and/or anti-foam agents may be employed in the separation zone, e.g., in the units of the separation zone. The inhibitors may be used to reduce the potential for fouling caused by polymerization of acrylates. The anti-foam agents may be used to reduce potential for foaming in the various streams of the separation zone. The polymerization inhibitors and/or the anti-foam agents may be used at one or more locations in the separation zone.

Returning to FIG. 2, at least a portion of stream 240 is directed to second column 246. Second column 246 separates the at least a portion of stream 240 into a distillate in line 248 and a residue in line 250. The distillate may be refluxed and the residue may be boiled up as shown. The distillate comprises at least 1 wt % alkylenating agent. Stream 248, like stream 240, may be considered an alkylenating agent stream. The second column residue exits second column 246 in line 250 and comprises a significant portion of acetic acid. At least a portion of line 250 may be returned to first column 244 for further separation. In one embodiment, at least a portion of line 250 is returned, either directly or indirectly, to reactor 206. Exemplary compositional ranges for the distillate and residue of second column 246 are shown in Table 5. Components other than those listed in Table 5 may also be present in the residue and distillate.

TABLE 5
SECOND COLUMN (246)

| | Conc. (wt. %) | Conc. (wt. %) | Conc. (wt. %) |
|---|---|---|---|
| Distillate | | | |
| Acrylic Acid | 0.01 to 10 | 0.05 to 5 | 0.1 to 0.5 |
| Acetic Acid | 10 to 50 | 20 to 40 | 25 to 35 |
| Water | 35 to 75 | 45 to 65 | 50 to 60 |
| Alkylenating Agent | at least 1 | 1 to 75 | 10 to 20 |
| Propionic Acid | 0.01 to 10 | 0.01 to 5 | 0.01 to 0.05 |
| Residue | | | |
| Acrylic Acid | 0.1 to 25 | 0.05 to 15 | 1 to 10 |
| Acetic Acid | 40 to 80 | 50 to 70 | 55 to 65 |
| Water | 1 to 40 | 5 to 35 | 10 to 30 |
| Alkylenating Agent | at least 1 | 1 to 75 | 10 to 20 |
| Propionic Acid | <10 | 0.001 to 5 | 0.001 to 1 |

In cases where any of the alkylenating agent split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 300 kPa, e.g., from 10 kPa to 100 kPa or from 40 kPa to 80 kPa. In preferred embodiments, the pressure at which the column(s) are operated is kept at a low level e.g., less than 100 kPa, less than 80 kPa, or less than 60 kPa. In terms of lower limits, the column(s) may be operated at a pressures of at least 1 kPa, e.g., at least 20 kPa or at least 40 kPa. Without being bound by theory, it is believed that alkylenating agents, e.g., formaldehyde, may not be sufficiently volatile at lower pressures. Thus, maintenance of the column pressures at these levels surprisingly and unexpectedly provides for efficient separation operations. In addition, it has surprisingly and unexpectedly been found that be maintaining a low pressure in the columns of alkylenating agent split unit 232 may inhibit and/or eliminate polymerization of the acrylate products, e.g., acrylic acid, which may contribute to fouling of the column(s).

In one embodiment, the alkylenating agent split is achieved via one or more liquid-liquid extraction units. Preferably, the one or more liquid-liquid extraction units employ one or more extraction agents. Multiple liquid-liquid extraction units may be employed to achieve the alkylenating agent split. Any suitable liquid-liquid extraction devices used for multiple equilibrium stage separations may be used. Also, other separation devices, e.g., traditional columns, may be employed in conjunction with the liquid-liquid extraction unit(s).

In one embodiment (not shown), the crude product stream is fed to a liquid-liquid extraction column where the crude product stream is contacted with an extraction agent, e.g., an organic solvent. The liquid-liquid extraction column extracts the acids, e.g., acrylic acid and acetic acid, from the crude product stream. An aqueous stage comprising water, alkylenating agent, and some acetic acid exits the liquid-liquid extraction unit. Small amounts of acylic acid may also be present in the aqueous stream. The aqueous phase may be further treated and/or recycled. An organic phase comprising acrylic acid, acetic acid, and the extraction agent also exits the liquid-liquid extraction unit. The organic phase may also comprise water and formaldehyde. The acrylic acid may be separated from the organic phase and collected as product. The acetic acid may be separated then recycled and/or used elsewhere. The solvent may be recovered and recycled to the liquid-liquid extraction unit.

The inventive process further comprises the step of separating the intermediate acrylate product stream to form a finished acrylate product stream and a first finished acetic acid stream. The finished acrylate product stream comprises acrylate product(s) and the first finished acetic acid stream comprises acetic acid. The separation of the acrylate products from the intermediate product stream to form the finished acrylate product may be referred to as the “acrylate product split.”

Returning to FIG. 2, intermediate acrylate product stream in line 242 exits alkylenating agent split unit 232 and is directed acrylate product split unit 234 for further separation, e.g., to further separate the acrylate products therefrom. Acrylate product split unit 234 may comprise any suitable separation device or combination of separation devices. Preferably, acrylate product split unit 234 comprises an azeotropic distillation column and optionally one or more additional separation devices. For example, acrylate product split unit 234 may comprise at least one column, e.g., a standard distillation column, an extractive distillation column, an azeotropic distillation column, and/or a liquid/liquid extraction column. In other embodiments, acrylate product split unit 234 comprises a precipitation unit, e.g., a crystallizer and/or a chiller. Preferably, acrylate product split unit 234 comprises an azeotropic distillation column and two standard distillation columns as shown in FIG. 2. In another embodiment, acrylate product split unit 234 comprises a liquid-liquid extraction unit. Of course, other suitable separation devices may be employed either alone or in combination with the devices mentioned herein.

In FIG. 2, acrylate product split unit 234 comprises third column 252, fourth column 254 and fifth column 256. Third column 252 is an azeotropic distillation column that utilizes an entrainer. Third column 252 receives at least a portion of intermediate acrylate product stream in line 242 and separates same in azeotropic distillation column 252 into a third residue in line 258 and a third distillate in line 260. The third residue exits azeotropic distillation column 252 in line 258 and comprises a significant portion of acrylate products. As such, stream in line 258 may be considered an acrylate product stream. The third distillate exits azeotropic distillation column 252 in line 260 and comprises a significant portion of entrainer. As such stream 128 may be considered an entrainer stream. The acrylate product stream in line 258 may be fed to fourth column 254 to yield a finished acrylate product stream 264. Entrainer stream in line 260 contains acetic acid and the stream may be further separated in fifth column 256 to recover a purified entrainer stream in line 270 and a purified acetic acid stream in line 268. The distillate may be refluxed and the residue may be boiled up as shown. The intermediate acrylate product stream in line 242 is introduced in the upper part of azeotropic distillation column 252, e.g., upper half or upper third. An entrainer, as described above, is also introduced to azeotropic distillation column 252 via line 262 to aid with the separation of acrylic acid from acetic acid. Depending on the type of entrainer, entrainer in line 262 may be introduced above or below the feed point of intermediate acrylate product stream 242. In another embodiment, the entrainer may be mixed with intermediate acrylate product stream 242 and co-fed to azeotropic distillation column 252.

The amount of entrainer fed to azeotropic distillation column 252 may vary widely depending on the concentration of acrylic acid in acrylate product stream 264. In terms of mass ratios of entrainer to crude acrylate products fed to the azeotropic distillation column 252 may be less than about 1:2, e.g., less than about 1:3 or less than about 1:10. When column conditions are controlled, as the amount of entrainer increases the amount of leakage of the entrainer into the third residue in line 258 may also increase. Thus, it is desirable to use an entrainer such that the leakage of the entrainer is less than 5 wt. %, e.g., less than 1 wt. % or less than 0.5 wt. %. Higher concentrations of entrainers may be tolerated in some embodiments.

In azeotropic distillation column 252, the majority of acrylic acid is removed from intermediate acrylate product stream 242 and is withdrawn, preferably continuously, as a residue in line 258. For example, the residue stream comprises at least 90% of the acrylic acid in the intermediate acrylate products stream 242, e.g., at least 95% of the acrylic acid or at least 98% of the acrylic acid. Azeotropic distillation column 252 also forms an overhead distillate (third distillate), which is withdrawn in line 260, and which may be condensed and refluxed, for example, at a ratio of from 30:1 to 1:30, e.g., from 25:1 to 1:25 or from 1:5 to 5:1. The third distillate in line 260 comprises acetic acid, alkylenating agent and the entrainer. When column 260 is operated at 40 kPa, the temperature of the third distillate existing in line 260 preferably is from 60° C. to 95° C., e.g., from 70° C. to 90° C. or from 80° C. to 85° C. The third residue in line 258 may comprise acrylic acid, entrainer, and acetic acid. The temperature of the third residue exiting in line 258 preferably is from 100° C. to 160° C., e.g., from 110° C. to 145° C. or from 120° C. to 135° C. In some embodiments, the pressure of entrainer column 252 may range from 0.1 kPa to 510 kPa, e.g., from 1 kPa to 475 kPa or from 1 kPa to 375 kPa. In an embodiment, the azeotropic distillation is carried out at a temperature lower than 115° C., e.g., lower than 110° C. or lower than 105° C. Exemplary compositional ranges for the distillate and residue of entrainer column 252 are shown in Table 6. Components other than those listed in Table 6 may also be present in the residue and distillate.

TABLE 6
ENTRAINER COLUMN (252)

| | Conc. (wt. %) | Conc. (wt. %) | Conc. (wt. %) |
|---|---|---|---|
| Distillate | | | |
| (Entrainer Stream) | | | |
| Acrylic Acid | 0.001 to 10 | 0.001 to 5 | less than 5 |
| Acetic Acid | 5 to 65 | 15 to 55 | 20 to 50 |
| Water | 0.01 to 20 | 0.01 to 10 | 0.01 to 5 |
| Alkylenating Agent | at least 85 | at least 90 | at least 95 |
| Propionic Acid | less than 10 | 0.001 to 5 | less than 1 |
| Entrainer | 30 to 99 | 40 to 85 | 50 to 80 |
| Residue (Acrylate | | | |
| Product Stream) | | | |
| Acrylic Acid | at least 5 | 5 to 99 | 35 to 90 |
| Acetic Acid | less than 50 | 5 to 40 | 5 to 20 |
| Water | less than 25 | 0.1 to 10 | less than 0.1 |
| Alkylenating Agent | less than 1 | less than 0.5 | less than 0.1 |
| Propionic Acid | less than 10 | 0.01 to 5 | 0.01 to 1 |
| Entrainer | 1 to 30 | 1 to 20 | 1 to 10 |

As mentioned above, the crude product stream of the present invention comprises little, if any, furfural and/or acrolein. As such the derivative stream(s) of the crude product streams will comprise little, if any, furfural and/or acrolein. In one embodiment, the derivative stream(s), e.g., the streams of the purification zone, comprises less than less than 500 wppm acrolein, e.g., less than 100 wppm, less than 50 wppm, or less than 10 wppm. In one embodiment, the derivative stream(s) comprises less than less than 500 wppm furfural, e.g., less than 100 wppm, less than 50 wppm, or less than 10 wppm.

In one embodiment, the third distillate in line 260, i.e., the entrainer stream, comprises entrainer, unreacted acetic acid, and alkylenating agent. For example, the entrainer stream may comprise at least 30 wt. % entrainer, e.g., at least 40 wt. %, at least 50 wt. %, or at least 60 wt. %. The entrainer stream may comprise at least 5 wt. % acetic acid, e.g., at least 15 wt. %, at least 25 wt. % or at least 35 wt. % The entrainer stream may also comprise substantially all of the alkylenating agent in the intermediate product stream. For example, the entrainer stream may comprise at least 85% of the alkylenating agent, e.g., at least 90%, at least 95%, at least 99% or at least 99.5%. The entrainer stream may comprise a small amount of acrylate product. For example, the entrainer stream may comprise at most 5 wt. % acrylate product, at most 3 wt. %, at most 1 wt. % or at most 0.8 wt. %.

In other embodiments, the acrylate product stream in line 258 comprises acrylate products, e.g., acrylic acid. In one embodiment, the acrylate product stream in line 258 comprises a significant portion of acrylic acid. The acrylate product stream in line 258, in one embodiment, comprises little if any alkylenating agent. For example, the acrylate product stream in line 258 may comprise less than 1 wt. % alkylenating agent, e.g., less than 0.1 wt. % alkylenating agent, less than 0.05 wt. %, or less than 0.01 wt. %. In addition to the acrylate products, the intermediate acrylate products stream optionally comprises acetic acid, water, propionic acid and other components.

To recover purified acrylic acid, the residue in line 258 may be further separated. In an embodiment of the present invention, the residue in line 258 is further separated in fourth column 254, also known as a “product recovery column.” In FIG. 2, the third residue in line 258 is introduced to fourth column 254, preferably in the top part of column 254, e.g., top half or top third. Fourth column 254 yields a fourth residue in line 264 comprising purified acrylic acid, which may be considered a “finished acrylate product stream,” and a fourth distillate in line 266 comprising entrainer and acetic acid. Fourth column 254 may be a tray column or packed column. In one embodiment, fourth column 254 is a tray column having from 5 to 150 trays, e.g., from 15 to 50 trays or from 20 to 45 trays. Although the temperature and pressure of fourth column 254 may vary, when at atmospheric pressure the temperature of the fourth residue exiting in line 264 from fourth column 254 preferably is from 95° C. to 150° C., e.g., from 100° C. to 135° C. or from 115° C. to 125° C. The temperature of the fourth distillate exiting in line 266 from fourth column 254 preferably is from 60° C. to 135° C., e.g., from 75° C. to 120° C. or from 90° C. to 110° C. The pressure of fourth column 254 may range from 0.1 kPa to 510 kPa, e.g., from 1 kPa to 475 kPa or from 1 kPa to 375 kPa. Exemplary components for the distillate and residue compositions for fourth column 254, i.e., product recovery column, are provided in Table 7 below. It should be understood that the distillate and residue may also contain other components, not listed in Table 7.

TABLE 7
ACRYLATE PRODUCT RECOVERY COLUMN (254)

| | Conc. (wt. %) | Conc. (wt. %) | Conc. (wt. %) |
|---|---|---|---|
| Distillate | | | |
| Acrylic Acid | 5 to 80 | 10 to 70 | 20 to 60 |
| Acetic Acid | 5 to 80 | 10 to 70 | 20 to 60 |
| Water | less than | less than | less than |
| | 10 ppm | 100 ppm | 100 ppm |
| Alkylenating Agent | less than 1 ppb | less than 1 ppb | less than 1 ppb |
| Propionic Acid | 0.01 to 10 | 0.01 to 5 | Undetectable |
| Entrainer | 1 to 50 | 5 to 40 | 10 to 35 |
| Residue | | | |
| (Finished Acrylate | | | |
| Product Stream) | | | |
| Acrylic Acid | 60 to 99 | 70 to 97 | 80 to 95 |
| Acetic Acid | 1 to 25 | 1 to 20 | 5 to 15 |
| Water | trace amount | trace amount | trace amount |
| Alkylenating Agent | trace amount | trace amount | trace amount |
| Propionic Acid | less than 10 | 0.001 to 5 | 0.001 to 1 |
| Entrainer | 1 to 45 | 1 to 35 | 1 to 25 |

In an embodiment, the fourth residue in line 264 comprises little, if any, alkylenating agent. In one embodiment, the fourth residue in line 264 may contain no alkylenating agent. For example, the fourth residue comprises less than 1 wt. % alkylenating agent, e.g., less than 0.1 wt. %, or less than 0.05 wt. %. In another embodiment, the fourth residue in line 264 comprises acetic acid. For example, the fourth residue in line 264 comprises less than 25 wt. % acetic acid, e.g., less than 20 wt. %, less than 15 wt. % or less than 10 wt. %. In one embodiment, the fourth residue may be further purified to recover a high purity acrylic acid product, i.e., 99 wt. % acrylic acid product stream.

In some embodiments, depending on the entrainer employed, minor amounts of the entrainer may be leaked into the third residue and concentrated with the fourth residue in line 264. The concentration of the entrainer in the fourth residue may be low, e.g., less than 10 wt. % based on the total weight of the fourth residue, or less than 5 wt. %. The majority of the entrainer may be removed in the distillate stream in line 266 and may be concentrated with acetic acid. In one embodiment, it may be necessary to further separate entrainer from acetic acid. In some embodiments, the entrainer may be separated and reused in entrainer column 252.

In an embodiment, the fourth distillate in line 266 comprises acetic acid and entrainer. In an embodiment, at least a portion of distillate stream in line 266 may be combined with the distillate stream in line 260 for further separation. In one embodiment, the distillate in line 266 may be further separated without being combined with the distillate in line 260.

As shown in FIG. 2, the third distillate in line 260 and the fourth distillate in line 266 are combined to form combined distillate stream 272, which is fed to fifth distillation column 256, which may be referred to as an “entrainer recovery column.” The combined distillate in line 272 is introduced to fifth column 256, preferably in the top part of column 256, e.g., top half or top third. In another embodiment, the third distillate in line 260 and the fourth distillate in line 266 are fed to the fifth distillation column 256 separately. For example, the fourth distillate in line 266 may be introduced to fifth column 256, preferably in the bottom part of column 256, e.g., bottom half or bottom third. For example, the third distillate in line 260 may be introduced to fifth column 256, preferably in the top part of column 256, e.g., top half or top third.

Fifth column 256 yields a fifth residue comprising acetic acid in line 268 and a fifth distillate comprising entrainer in line 270. Exemplary components for the distillate and residue compositions for fifth column 256 are provided in Table 8 below. It should be understood that the distillate and residue may also contain other components, not listed in Table 8.

TABLE 8
ENTRAINER RECOVERY COLUMN (256)

| | Conc. (wt. %) | Conc. (wt. %) | Conc. (wt. %) |
|---|---|---|---|
| Distillate | | | |
| Acrylic Acid | less than 10 | less than 5 | less than 1 |
| Acetic Acid | 1 to 50 | 1 to 30 | 1 to 20 |
| Water | 0.01 to 30 | 0.01 to 20 | 1 to 10 |
| Alkylenating Agent | at least 0.01 | at least 0.01 | at least 0.01 |
| Propionic Acid | 0.01 to 10 | 0.01 to 5 | Undetectable |
| Entrainer | 15 to 99 | 30 to 97 | 50 to 95 |
| Residue | | | |
| Acrylic Acid | 1 to 30 | 1 to 20 | 1 to 15 |
| Acetic Acid | 10 to 95 | 30 to 90 | 40 to 80 |
| Water | less than 1 | less than 1 | less than 1 |
| Alkylenating Agent | less than 5 | less than 3 | less than 1 |
| Propionic Acid | less than 10 | 0.001 to 5 | 0.001 to 1 |
| Entrainer | 1 to 60 | 10 to 50 | 20 to 40 |

Fifth column 256 may be a tray column or packed column. In one embodiment, fifth column 256 is a tray column having from 5 to 150 trays, e.g., from 15 to 50 trays or from 20 to 45 trays. Although the temperature and pressure of fifth column 256 may vary, when at atmospheric pressure the temperature of the fifth residue exiting in line 268 from fifth column 256 preferably is from 60° C. to 130° C., e.g., from 70° C. to 115° C. or from 85° C. to 95° C. The temperature of the fifth distillate exiting in line 270 from fifth column 256 preferably is from 40° C. to 90° C., e.g., from 50° C. to 80° C. or from 60° C. to 70° C. The pressure of fifth column 256 may range from 0.1 kPa to 510 kPa, e.g., from 1 kPa to 475 kPa or from 1 kPa to 375 kPa.

In one embodiment, entrainer in line 270 may be returned and recycled to azeotropic distillation column 252. In one embodiment, entrainer in line 270 may be combined with fresh entrainer in line 262 before returning to azeotropic distillation column 252. In some embodiments, entrainer in line 270 may be returned to azeotropic distillation column 252 without being combined with fresh entrainer. In one embodiment, acetic acid in line 268 may be returned and recycled, directly or indirectly, to reactor 206. Acetic acid in line 268 may be combined with fresh acetic acid in line 208 before returning to reactor 206.

The inventive process further comprises the step of separating an alkylenating agent stream to form a purified alkylenating stream and a purified acetic acid stream. The purified alkylenating agent stream comprises a significant portion of alkylenating agent, and the purified acetic acid stream comprises acetic acid and water. The separation of the alkylenating agent from the acetic acid may be referred to as the “acetic acid split.”

Returning to FIG. 2, alkylenating agent stream 248 exits alkylenating agent split unit 232 and is directed to acetic acid split unit 236 for further separation, e.g., to further separate the alkylenating agent and the acetic acid therefrom. Acetic acid split unit 236 may comprise any suitable separation device or combination of separation devices. For example, acetic acid split unit 236 may comprise at least one column, e.g., a standard distillation column, an extractive distillation column and/or an azeotropic distillation column. In other embodiments, acetic acid split unit 236 comprises a precipitation unit, e.g., a crystallizer and/or a chiller. Preferably, acetic acid split unit 236 comprises a standard distillation column as shown in FIG. 2. In another embodiment, acetic acid split unit 236 comprises a liquid-liquid extraction unit. Of course, other suitable separation devices may be employed either alone or in combination with the devices mentioned herein.

In FIG. 2, acetic acid split unit 236 comprises sixth column 274. Acetic acid split unit 236 receives at least a portion of alkylenating agent stream in line 248 and separates same into a sixth distillate comprising alkylenating agent in line 278, e.g., a purified alkylenating stream, and a sixth residue comprising acetic acid in line 280, e.g., a purified acetic acid stream. The distillate may be refluxed and the residue may be boiled up as shown. In one embodiment, at least a portion of line 278 and/or line 280 are returned, either directly or indirectly, to reactor 206. At least a portion of stream in line 280 may be further separated. In another embodiment, at least a portion of the acetic acid-containing stream in line 280 may be directed to an ethanol production system that utilizes the hydrogenation of acetic acid form the ethanol. In another embodiment, at least a portion of the acetic acid-containing stream in either or both of lines 268 and 266 may be directed to a vinyl acetate system that utilizes the reaction of ethylene, acetic acid, and oxygen form the vinyl acetate.

The stream in line 278 comprises alkylenating agent and water. The stream in line 280 comprises acetic acid and water. Exemplary compositional ranges for the distillate and residue of sixth column 274 are shown in Table 9. Components other than those listed in Table 9 may also be present in the residue and distillate.

TABLE 9
ACETIC ACID RECOVERY COLUMN (274)

| | Conc. (wt. %) | Conc. (wt. %) | Conc. (wt. %) |
|---|---|---|---|
| Distillate | | | |
| Acrylic Acid | less than 1 | 0.001 to 5 | 0.001 to 1 |
| Acetic Acid | less than 1 | 0.001 to 5 | 0.001 to 1 |
| Water | 40 to 80 | 50 to 70 | 55 to 65 |
| Alkylenating Agent | 20 to 60 | 30 to 50 | 35 to 45 |
| Propionic Acid | less than 1 | 0.001 to 5 | 0.001 to 1 |
| Residue | | | |
| Acrylic Acid | less than 1 | 0.01 to 5 | 0.1 to 1 |
| Acetic Acid | 25 to 65 | 35 to 55 | 40 to 50 |
| Water | 35 to 75 | 45 to 65 | 50 to 60 |
| Alkylenating Agent | less than 1 | 0.01 to 5 | 0.1 to 1 |
| Propionic Acid | less than 1 | 0.001 to 5 | 0.001 to 1 |

In cases where the acetic acid split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 300 kPa.

The inventive process further comprises the step of separating the purified acetic acid stream to form a second finished acetic acid stream and a water stream. The second finished acetic acid stream comprises a major portion of acetic acid, and the water stream comprises mostly water. The separation of the acetic from the water may be referred to as dehydration.

Returning to FIG. 2, sixth residue 280 exits acetic acid split unit 236 and is directed to drying unit 238 for further separation, e.g., to remove water from the acetic acid. Drying unit 238 may comprise any suitable separation device or combination of separation devices. For example, drying unit 238 may comprise at least one column, e.g., a standard distillation column, an extractive distillation column and/or an azeotropic distillation column. In other embodiments, drying unit 238 comprises a dryer and/or a molecular sieve unit. In a preferred embodiment, drying unit 238 comprises a liquid-liquid extraction unit. In one embodiment, drying unit 238 comprises a standard distillation column as shown in FIG. 2. Of course, other suitable separation devices may be employed either alone or in combination with the devices mentioned herein.

In FIG. 2, drying unit 238 comprises seventh column 276. Drying unit 238 receives at least a portion of second finished acetic acid stream in line 280 and separates same into a seventh distillate comprising a major portion of water in line 282 and a seventh residue comprising acetic acid and small amounts of water in line 284. The distillate may be refluxed and the residue may be boiled up as shown. In one embodiment, at least a portion of line 284 is returned, either directly or indirectly, to reactor 206. In another embodiment, at least a portion of the acetic acid-containing stream in line 284 may be directed to an ethanol production system that utilizes the hydrogenation of acetic acid form the ethanol. In another embodiment, at least a portion of the acetic acid-containing stream in either or both of lines 268 and 266 may be directed to a vinyl acetate system that utilizes the reaction of ethylene, acetic acid, and oxygen form the vinyl acetate.

Exemplary compositional ranges for the distillate and residue of seventh column 276 are shown in Table 10. Components other than those listed in Table 10 may also be present in the residue and distillate.

TABLE 10
DRYING COLUMN (276)

| | Conc. (wt. %) | Conc. (wt. %) | Conc. (wt. %) |
|---|---|---|---|
| Distillate | | | |
| Acrylic Acid | less than 1 | 0.001 to 5 | 0.001 to 1 |
| Acetic Acid | less than 1 | 0.01 to 5 | 0.01 to 1 |
| Water | 90 to 99.9 | 95 to 99.9 | 95 to 99.5 |
| Alkylenating Agent | less than 1 | 0.01 to 5 | 0.01 to 1 |
| Propionic Acid | less than 1 | 0.001 to 5 | 0.001 to 1 |
| Residue | | | |
| Acrylic Acid | less than 1 | 0.01 to 5 | 0.01 to 1 |
| Acetic Acid | 75 to 99.9 | 85 to 99.5 | 90 to 99.5 |
| Water | 25 to 65 | 35 to 55 | 40 to 50 |
| Alkylenating Agent | less than 1 | less than 0.001 | less than 0.0001 |
| Propionic Acid | less than 1 | 0.001 to 5 | 0.001 to 1 |

In cases where the drying unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 300 kPa. FIG. 2 also shows tank 286, which, collects at least one of the process streams prior to recycling same to reactor 206. Tank 286 is an optional feature. The various recycle streams that may, alternatively, be recycled directly to reactor 206 without being collected in tank 286.

### EXAMPLES

### Example 1

A partial simulation of a process in accordance with the embodiment of FIG. 2 was conducted using ASPEN™ software. The simulation utilized a crude product stream as discussed above. The compositions of the process streams of acrylate product split unit 234 are shown in Table 11.

TABLE 11
SIMULATED COMPOSITIONAL DATA FOR
ACRYLATE PRODUCT SPLIT PROCESS STREAMS

| | Azeotropic | | | | | |
|---|---|---|---|---|---|---|
| | Distillation | | Product Recovery | | Entrainer Recovery | |
| | Column 252 | | Column 254 | | Column 256 | |
| | (wt. %) | | (wt. %) | | (wt. %) | |
| Composition | Distillate | Residue | Distillate | Residue | Distillate | Residue |
| Acrylic Acid | 0.8 | 80.8 | 43.7 | 85.5 | 0 | 7.5 |
| Acetic Acid | 35.0 | 11.5 | 34.1 | 8.7 | 9.9 | 59.9 |
| Water | 3.4 | 0.0 | 0.0 | 0.0 | 6.3 | 0.0 |
| Alkylenating Agent | 0.3 | 0.0 | 0.0 | 0.0 | 0.6 | 0.0 |
| Entrainer (Toluene) | 60.5 | 6.6 | 21.5 | 4.7 | 83.3 | 32.5 |

As shown by the simulation, an intermediate product stream comprising acrylic acid and acetic acid can be effectively separated in accordance with the present invention to achieve a finished acrylic acid product comprising over 85 wt. % acrylic acid.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing an acrylate product, comprising the steps of:
providing a crude product stream comprising the acrylate product, acetic acid and an alkylenating agent;
separating at least a portion of the crude product stream into an intermediate acrylate product stream and an alkylenating stream, wherein the intermediate acrylate product stream comprises acrylate product;
separating at least a portion of the intermediate acrylate product stream by azeotropic distillation in the presence of an entrainer to form a first distillate comprising entrainer and acetic acid and a first residue comprising acrylate product, entrainer, and acetic acid; and
recovering acrylate product from the first residue.

2. The process of claim 1, wherein the first distillate comprises at least 5 wt. % of the acetic acid.

3. The process of claim 1, wherein the first residue comprises at least 90 wt. % of the acrylate product.

4. The process of claim 1, wherein the intermediate acrylate product stream comprises less than 1 wt. % alkylenating agent.

5. The process of claim 1, further comprising purifying the acrylate product stream to recover pure acrylic acid.

6. The process of claim 1, wherein the entrainer is selected from a group consisting of methyl-isobutyl ketone, di-isobutyl ketone, o-xylene, p-xylene, benzene, n-hexane, cyclohexane, isopropyl-alcohol, diphenyl ether, biphenyl, n-butylacetate, and i-butylacetate.

7. The process of claim 1, wherein the entrainer is toluene.

8. The process of claim 1, wherein the alkylenating comprises formaldehyde.

9. The process of claim 1, wherein the azeotropic distillation is carried out at a reboiler temperature from 100° C. to 160° C.

10. The process of claim 1, wherein the azeotropic distillation is carried out at a temperature lower than 115° C.

11. The process of claim 1, wherein the first residue is substantially free of the alkylenating agent.

12. The process of claim 1, further comprising separating at least a portion of the first residue in a second column into a second distillate comprising entrainer and acetic acid and a second residue comprising acrylate product.

13. The process of claim 12, wherein the second residue comprises from 60 to 99 wt. % acrylate product.

14. The process of claim 12, wherein the second residue comprises at least 10 wt. % acrylate product.

15. The process of claim 12, wherein the second residue comprises less than 1 wt. % alkylenating agent.

16. The process of claim 12, wherein the second residue comprises less than 15 wt. % acetic acid.

17. The process of claim 12, further comprising separating at least a portion of the second distillate in a third distillation column to recover entrainer.

18. The process of claim 17, further comprising separating at least a portion of the first distillate in the third distillation column into a third distillate comprising entrainer and a third residue comprising acetic acid.

19. The process of claim 18, wherein the third distillate comprises less than 30 wt. % acetic acid.

20. The process of claim 18, wherein the third distillate comprises from 50 to 95 wt. % entrainer.

21. The process of claim 18, further comprising returning at least a portion of the third distillate to the first distillation column.

22. The process of claim 1, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil petroleum, coal, biomass, and combinations thereof.

23. A process for producing an acrylate product, comprising the steps of:
alkylenating acetic acid from an acetic acid feed stream in a reactor to form a crude product stream comprising the acrylate product, an alkylenating agent and acetic acid;
separating the crude product stream into an intermediate acrylate product stream and an alkylenating stream, wherein the intermediate acrylate product stream comprises acrylate product;
separating at least a portion of the intermediate acrylate product stream using azeotropic distillation in a first column in the presence of one or more entrainers to form a first distillate comprising entrainer and acetic acid, and a first residue comprising acrylate product, entrainer, and acetic acid;
separating at least a portion of the first residue in a second column to form a second distillate comprising acetic acid and entrainer and a second residue comprising acrylate product; and
recovering acrylate product from the second residue.

24. The process of claim 23, wherein the entrainer is toluene.

25. The process of claim 23, wherein the second residue comprises less than 25 wt. % of the entrainer.

26. The process of claim 23, wherein the second residue comprises from 60 to 99 wt. % acrylate product.

27. The process of claim 23, further comprising separating at least a portion of the first distillate and at least a portion of the second distillate in a third distillation column to form a third distillate comprising entrainer and a third residue comprising acetic acid.

28. The process of claim 27, further comprising combining the at least a portion of the first distillate and the at least a portion of the second distillate prior to separating entrainer in the third distillation column.

29. The process of claim 27, further comprising recovering entrainer from the third distillate and returning the entrainer to the first distillation column.

30. A process for producing an acrylate product, comprising the steps of:
providing a crude product stream comprising the acrylate product, acetic acid and an alkylenating agent;
separating at least a portion of the crude product stream by azeotropic distillation in the presence of an entrainer to form a first distillate comprising entrainer and acetic acid and a first residue comprising acrylate product, entrainer, and acetic acid; and
recovering acrylate product from the first residue.

31. A process for producing an acrylate product, comprising the steps of:
providing a crude product stream comprising the acrylate product and an alkylenating agent;
diluting the crude product stream with one or more entrainers to prevent the polymerization of the acrylate product;
separating at least a portion of the diluted crude product stream; and
recovering the acrylate product, wherein the acrylate product comprises less than 5 wt. % alkylenating agent.
